(19) United States
(12) Patent Application Publication
(10) Pub. No.: US 2026/0207675 A1
(43) Pub. Date: Jul. 23, 2026

(54) CELL COMPOSITION

(71) Applicant: Interstem Co., Ltd., Tokyo (JP)

(72) Inventors: Akihiro Horii, Hachioji-shi (JP); Yoshihito Tachi, Hachioji-shi (JP)

(21) Appl. No.: 19/142,247

(22) PCT Filed: Dec. 27, 2023
(86) PCT No.: PCT/JP2023/046873

§ 371 Date: Jun. 23, 2025

(30) Foreign Application Priority Data
Dec. 28, 2022 (JP) 2022-211906

(51) Int. Cl. A61K 35/32; A61K 9/06; A61K 35/28; A61K 47/20; A61K 47/42; C12N 5/00; C12N 5/077; C12N 5/0775

(52) U.S. Cl. CPC A61K 35/32; A61K 9/06; A61K 35/28; A61K 47/20; A61K 47/42; C12N 5/0655; C12N 5/0662; C12N 5/562

(57) ABSTRACT

The present invention provides a cell composition in which a cell is suspended in a solution containing 5.0 v/v % or greater and 10.0 v/v % or less of dimethyl sulfoxide and 2.0 w/v % or greater and 5.0 w/v % or less of albumin, and a method for producing a hydrogel cell composition, including a step of mixing a cell composition containing dimethyl sulfoxide, albumin, and a cell, fibrinogen, and thrombin to prepare a hydrogel cell composition, in which the cell composition contains 5.0 v/v % or greater and 10.0 v/v % or less of dimethyl sulfoxide, 2.0 w/v % or greater and 5.0 w/v % or less of albumin, and one or more cells selected from the group consisting of a cartilage cell and an adipose-derived mesenchymal stem cell.

### TECHNICAL FIELD

The present invention relates to a cell composition prepared with a cryopreservation agent containing DMSO, and a pharmaceutical composition suitable for transplantation, which contains the cell composition.

Priority is claimed on Japanese Patent Application No. 2022-211906, filed Dec. 28, 2022, the content of which is incorporated herein by reference.

### BACKGROUND ART

In recent years, efforts have been made in regenerative medicine in which cells are considered as pharmaceuticals and administered. In the regenerative medicine, there is a case where the administered cells are expected to continuously remain in the body, and there is also a case where the administered cells only cause an initial biological reaction and do not need to remain in the body. For example, mesenchymal stem cells do not continuously remain in the administered target, but are expected to suppress the pathological condition exhibited by the excessive immunity. In addition, the regenerative medicine also includes repairing epithelial tissues or cartilage tissues that are deficient due to external injuries or diseases using epithelial cells or cartilage cells. In such regenerative medicine, cells that are cultured and prepared in vitro are used. Since sterile production is required for the preparation of cells, the cells are prepared in a dedicated facility outside a medical institution in many cases. The cells prepared for regenerative medicine are usually refrigerated or stored in a refrigerator in order to suppress changes in the cells immediately after the production, and are stored until transplantation. In order to stably store cells for a longer period of time, it is preferable that the cells are stored by freezing rather than being stored in a refrigerator. In this case, in order to reduce damage (cryoinjury) to cells in the freezing or in the subsequent thawing treatment, various cryopreservation agents have been developed.

Dimethyl sulfoxide (DMSO) is an extremely effective preservative in a frozen state or at a low temperature, and is an effective component of many cryopreservation agents. However, the cytotoxicity is strong in a temperature range of about the body temperature. Therefore, there is a problem in that a cell composition prepared with a cryopreservation agent containing DMSO needs to be avoided from being administered to the body of an animal as it is, or needs to be administered immediately after being thawed. Therefore, the cells for administration are transported to a medical institution in a frozen state, and are thawed and diluted with physiological saline or the like before surgery to be used for administration, or the cryopreservation agent is removed by a centrifugation treatment immediately after thawing so that the cells are used for administration as a cell suspension suspended in a solution containing no DMSO. In the latter case, since a dedicated sterile facility is required in a medical institution, the cells are thawed in an external institution in advance and may be refrigerated and transported to the medical institution in a state of a cell suspension suspended in a solution containing no DMSO. However, there is a concern of degradation of the functions of the cells during refrigerated transport in the method.

For example, Patent Document 1 discloses that damage to mesenchymal stem cells due to washing can be reduced by performing washing to remove trypsin during cell recovery with a bicarbonate Ringer's solution containing human serum albumin after culturing mesenchymal stem cells and that cryoinjury to mesenchymal stem cells can be reduced by using a freezing culture medium containing DMSO in a bicarbonate Ringer's solution containing human serum albumin. In addition, Patent Document 2 describes that cryoinjury to adipose tissue-derived mesenchymal stem cells can be reduced by using hydroxylethyl starch together with DMSO as an effective component. In addition, Patent Document 3 discloses a method for carrying out cryopreservation of a cell composition in which mesenchymal cells are suspended in a cryopreservation liquid containing 5 w/w % or greater and 10 w/w % or less of DMSO and containing 5 w/w % or greater and 10 w/w % or less of hydroxylethyl starch or 1 w/w % or greater and 5 w/w % or less of dextran.

### CITATION LIST

### Patent Documents

Patent Document 1: Japanese Patent No. 5394932
Patent Document 2: Japanese Patent No. 7072147
Patent Document 3: Japanese Patent No. 6512759

### SUMMARY OF INVENTION

### Technical Problem

An object of the present invention is to provide a cell composition having satisfactory cell survivability and satisfactory tissue adhesiveness after thawing, even though the cell composition is cryopreserved in the presence of DMSO, and a pharmaceutical composition containing the cell composition.

### Solution to Problem

The present inventors have found that a frozen substance of a cell composition obtained by suspending cartilage cells or adipose-derived mesenchymal stem cells with a cell cryopreservation agent containing 2.0 w/v % or greater and 5.0 w/v % or less of albumin together with 5.0 v/v % or greater and 10.0 v/v % or less of DMSO is thawed to obtain a cell composition having the cells with a satisfactory survival rate and a satisfactory tissue adhesion rate, thereby completing the present invention.

That is, the present invention provides the following aspects.
[1]A cell composition, in which a cell is suspended in a solution containing 5.0 v/v % or greater and 10.0 v/v % or less of dimethyl sulfoxide and 2.0 w/v % or greater and 5.0 w/v % or less of albumin.
[2] The cell composition according to [1], in which a concentration of the dimethyl sulfoxide in the solution is 5.0 v/v % or greater and 7.5 v/v % or less.
[3] The cell composition according to [1], in which a concentration of the dimethyl sulfoxide in the solution is 7.5 v/v %.
[4] The cell composition according to any one of [1] to [3], in which a concentration of the albumin in the solution is 2.5 w/v %.
[5] The cell composition according to any one of [1] to [4], in which the cell is frozen.
[6] The cell composition according to any one of [1] to [5], in which the cell is a cartilage cell or a mesenchymal stem cell.
[7] The cell composition according to any one of [1] to [6], in which the cell composition does not contain dextran.
[8] The cell composition according to any one of [11] to [7], in which the solution is a solution obtained by allowing a culture medium for cell culture, phosphate buffered saline, or a mixed solution thereof to contain dimethyl sulfoxide and albumin.
[9] The cell composition according to [8], in which the culture medium for cell culture is D'MEM.
[10] The cell composition according to any one of [1] to [9], in which the cell is suspended in D'MEM containing 5.0 v/v % or greater and 10.0 v/v % or less of dimethyl sulfoxide and 2.0 w/v % or greater and 5.0 w/v % or less of albumin.
[11] The cell composition according to any one of [1] to [10], in which a concentration of the cell in the cell composition is 2.5×10⁷ cells/mL or greater.
[12]A pharmaceutical composition including: the cell composition according to any one of [1] to [11].
[13] The pharmaceutical composition according to [12], further including: fibrinogen; and thrombin.
[14] The pharmaceutical composition according to [12], in which the cell composition is encapsulated in a hydrogel.
[15] The pharmaceutical composition according to [14], in which the hydrogel is a fibrin gel.
[16] The pharmaceutical composition according to any one of [12] to [15], in which the pharmaceutical composition is a composition for transplanting into a cartilage tissue.
[17]A kit for preparing a pharmaceutical composition, including: the cell composition according to any one of [1] to [11].
[18] The kit for preparing a pharmaceutical composition according to [17], further including: fibrinogen; and thrombin.
[19]A method for producing a hydrogel cell composition, including: a step of mixing a cell composition containing dimethyl sulfoxide, albumin, and a cell, fibrinogen, and thrombin to prepare a hydrogel cell composition, in which the cell composition contains 5.0 v/v % or greater and 10.0 v/v % or less of dimethyl sulfoxide, 2.0 w/v % or greater and 5.0 w/v % or less of albumin, and one or more cells selected from the group consisting of a cartilage cell and an adipose-derived mesenchymal stem cell.
[20] The method for producing a hydrogel cell composition according to [19], in which the cell composition does not contain dextran.
[21] The method for producing a hydrogel cell composition according to [19] or [20], in which the cell composition is formed such that the cell is suspended in a solution obtained by allowing a culture medium for cell culture, phosphate buffered saline, or a mixed solution thereof to contain dimethyl sulfoxide and albumin.
[22] The method for producing a hydrogel cell composition according to [21], in which the culture medium for cell culture is D'MEM.
[23] The method for producing a hydrogel cell composition according to any one of [19] to [22], in which the cell composition and the thrombin are mixed, and the obtained mixture is mixed with the fibrinogen.
[24] The method for producing a hydrogel cell composition according to any one of [19] to [23], in which a member which mixes two liquids, which includes a first injection port, a second injection port, a mixing portion that mixes a solution injected from the first injection port and a solution injected from the second injection port, and a discharge port that discharges contents of the mixing portion, is used to inject a mixture of the cell composition and the thrombin from the first injection port and inject the fibrinogen from the second injection port, and in the mixture, the mixture of the cell composition and the thrombin is mixed with the fibrinogen to prepare a hydrogel cell composition, and the hydrogel cell composition is discharged from the discharge port.
[25] The method for producing a hydrogel cell composition according to any one of [19] to [24], in which the cell composition is a frozen substance, and the hydrogel cell composition is prepared by thawing the cell composition, mixing the cell composition with thrombin, and mixing the obtained mixture with fibrinogen.
[26] The method for producing a hydrogel cell composition according to any one of [19] to [25], in which the cell composition is thawed within 5 minutes, and the hydrogel cell composition is transplanted into an animal after 20 minutes to 2 hours from the thawing of the cell composition.

### Advantageous Effects of Invention

According to the present invention, it is possible to provide a cell composition having satisfactory cell survivability and satisfactory tissue adhesiveness after thawing, even though the cell composition is cryopreserved in the presence of DMSO, and a pharmaceutical composition containing the cell composition.

### BRIEF DESCRIPTION OF DRAWINGS

FIG. 1 Stained images obtained by performing Safranin O staining after culturing a cell-containing fibrin gel, in which cartilage cells thawed after cryopreservation are encapsulated in a fibrin gel for 4 weeks in Example 4 (FIG. 1(A): cartilage matrix-stained enhanced image, FIG. 1(B): cell nucleus-enhanced image).

FIG. 2 Stained images obtained by culturing a cell-containing fibrin gel, in which adipose tissue-derived mesenchymal stem cells thawed after cryopreservation are encapsulated in a fibrin gel for 4 weeks and performing immunostaining with type I collagen and nuclear staining in Example 5 (FIG. 2(A): stained image with type I collagen, FIG. 2(B): stained image with hematoxylin in the same visual field).

FIG. 3 A Safranin O-stained image (FIG. 3(A)) and a HE-stained image (FIG. 3(B)) of a transplantation site captured 4 weeks after the transplantation of cartilage cells thawed after cryopreservation into a cartilage defect site of a cartilage defect model animal in a state of being encapsulated in a fibrin gel in Example 6.

FIG. 4 A fluorescence stained image of live cells/dead cells after encapsulating cartilage cells in each hydrogel and culturing the cells in Example 7 (FIG. 4(A): binarized black image of live cells in a fibrin gel, FIG. 4(B): binarized black image of dead cell nuclei in a fibrin gel, FIG. 4(C): binarized black image of live cells in a PVA-PEG gel, FIG. 4(D): binarized black image of dead cell nuclei in a PVA-PEG gel, FIG. 4(E): fluorescence image of live cells in a collagen gel, FIG. 4(F): binarized black image of dead cell nuclei in a collagen gel).

FIG. 5 Graphs showing the expression ratios of SOX9, COL2, and ACAN to GAPDH in cells after cryopreservation of cartilage cells of each subculture number, and thawing and culturing the cells in an alginate gel in Example 8 (FIG. 7(A): SOX9, FIG. 7(B): COL2, FIG. 7(C): ACAN).

### DESCRIPTION OF EMBODIMENTS

### <DMSO-Containing Cell Composition>

The cell composition according to the present invention is a cell composition in which cells are suspended in a solution containing 5.0 v/v % or greater and 10.0 v/v % or less of DMSO and 2.0 w/v % or greater and 5.0 w/v % or less of albumin. Since the DMSO concentration of the cell composition according to the present invention (hereinafter, also referred to as “DMSO-containing cell composition”) is 5.0 v/v % or greater and 10.0 v/v % or less and the albumin concentration of the cell composition is 2.0 w/v % or greater and 5.0 w/v % or less, damage to cells due to the freezing and the thawing treatment can be sufficiently suppressed.

The DMSO concentration of the DMSO-containing cell composition is 5.0 v/v % or greater and 10.0 v/v % or less, preferably 5.0 v/v % or greater and 7.5 v/v % or less, and particularly preferably 7.5 v/v %. In a case where the DMSO concentration is 5.0 v/v % or greater, damage to cells in the DMSO-containing cell composition due to the freezing can be sufficiently suppressed in a case where the DMSO-containing cell composition is cryopreserved. In addition, in a case where the DMSO concentration is 10.0 v/v % or less, damage to the cells in the DMSO-containing cell composition can be sufficiently suppressed after the DMSO-containing cell composition is subjected to the thawing treatment. The DMSO-containing cell composition can be safely administered to an animal as it is, or the DMSO concentration can be lowered simply by dilution and the DMSO-containing cell composition can be more safely administered to an animal. Therefore, the DMSO-containing cell composition is useful as an effective component of a pharmaceutical composition, and the DMSO-containing cell composition can also be used as a pharmaceutical composition. For example, a cell composition for transplantation which has cells with a satisfactory survival rate and a satisfactory tissue adhesion rate in a case of transplanting the DMSO-containing cell composition into an animal can be produced since the DMSO-containing cell composition contains cells for transplantation.

The albumin concentration of the DMSO-containing cell composition is 2.0 w/v % or greater and 5.0 w/v % or less, preferably 2.5 w/v % or greater and 5.0 w/v % or less, more preferably 2.5 w/v % or greater and 3.0 w/v % or less, and particularly preferably 2.5 w/v %. In a case where the albumin concentration thereof is in the above-described ranges, damage to cells in the composition due to the freezing and the thawing treatment can be sufficiently suppressed without the composition containing other additives. In a case where the albumin concentration of the DMSO-containing cell composition is 2.5 w/v % or greater and 3.0 w/v % or less, particularly in a case where the albumin concentration is 2.5 w/v %, a cell composition for transplantation which has cells with a particularly satisfactory survival rate and a particularly satisfactory tissue adhesion rate in a case of transplanting the DMSO-containing cell composition into an animal can be produced.

The albumin to be contained in the DMSO-containing cell composition is not particularly limited, but is preferably serum albumin and more preferably serum albumin of an animal to which a pharmaceutical composition to be produced from the DMSO-containing cell composition is to be administered. For example, a cell composition for transplantation which contains the same species of albumin is preferable to a cell composition for transplantation which contains a different species of albumin from the viewpoint of suppressing an immune reaction.

The cells contained in the DMSO-containing cell composition are not particularly limited, and may be cells collected from a living body, cultured cells, or cells subjected to various gene modification treatments such as knockdown or a knock-in treatment of a specific gene. Examples of the cells include mesenchymal cells such as fibroblasts, synovial cells, cartilage cells, and osteoblasts, or precursor cells thereof; immune cells such as vascular endothelial cells, neutrophils, eosinophils, basophils, monocytes, T cells, B cells, and dendritic cells; and myocardial cells, epithelial cells, keratinocytes, nerve cells, liver cells, hematopoietic stem cells, mesenchymal stem cells, embryonic stem cells, and induced pluripotent stem cells. Here, the mesenchymal stem cells are stem cells contained in the bone marrow, the adipose tissue, the placenta, the umbilical cord, the dental pulp, and the like.

The cartilage cells may be cells prepared by culturing primary cells isolated from cartilage tissues of animals, or may be cultured cells that have been established. The collection of cartilage tissues of animals, the recovery of cartilage cells from cartilage tissues, and the culture of recovered cartilage cells can be carried out by a method of the related art. The subculture number of the cartilage cells recovered from the cartilage tissues is not particularly limited, but is, for example, preferably 13 or more subcultures, 12 or more subcultures, 11 or more subcultures, 10 or more subcultures, 7 or more subcultures, or 4 or more subcultures. The subculture number thereof is more preferably 11 or more subcultures and still more preferably 11 subcultures.

The mesenchymal stem cells can be prepared by culturing primary cells isolated from the bone marrow, the adipose tissue, the placenta, the umbilical cord, and the dental pulp of an animal using a method of the related art.

In a case where the DMSO-containing cell composition is used as an effective component of a cell composition for transplantation, the cells for transplantation to be contained in the DMSO-containing cell composition are preferably one or more kinds of cells selected from the group consisting of cartilage cells and mesenchymal stem cells. By using these cells, in particular, a cell composition for transplantation with satisfactory tissue adhesiveness to cartilage tissues can be prepared. Further, even in a case where cells derived from other tissues or stem cells are used as the cells for transplantation, a cell composition for transplantation with a satisfactory survivability and satisfactory tissue adhesiveness can be prepared in the same manner as in a case where cartilage cells are used.

The cells to be contained in the DMSO-containing cell composition are not particularly limited as long as the cells are derived from an animal. The animal may be, for example, a mammal, a bird, a reptile, an amphibian, or fish. The cells to be contained in the DMSO-containing cell composition are preferably mammalian cells and particularly preferably human cells, but cells derived from an experimental animal, livestock, or a pet animal are also preferable. Examples of the experimental animal, the livestock, and the pet animal include a mouse, a rat, a rabbit, a guinea pig, a hamster, a monkey, a sheep, a horse, a cow, a pig, a donkey, a dog, and a cat.

In a case where the DMSO-containing cell composition is used as an effective component of a pharmaceutical composition, the cells to be contained in the DMSO-containing cell composition may be cells derived from individual animals that are targets for the administration of the pharmaceutical composition or cells derived from individual animals that are not targets for the administration. For example, a cell composition for autologous transplantation can be prepared from a DMSO-containing cell composition containing cells derived from individual animals that are targets for the administration of the pharmaceutical composition. In addition, a cell composition for allogeneic transplantation can be prepared from a DMSO-containing cell composition containing cells derived from individual animals of the same species other than the targets for administration of the pharmaceutical composition.

The DMSO-containing cell composition can be prepared, for example, by suspending cells in a solution obtained by allowing DMSO and albumin to be contained at a predetermined concentration in a solution in which cells can survive. The solution in which cells can survive is not particularly limited, and can be appropriately determined in consideration of the kind and the like of cells. Examples of the solution in which cells can survive include an isotonic solution, and examples of the isotonic solution include a culture medium for cell culture, phosphate buffered saline (PBS), physiological saline, a lactate Ringer's solution, a Ringer's solution, an acetate Ringer's solution, and a bicarbonate Ringer's solution. Among these, a culture medium for cell culture, PBS, or a mixed solution of a culture medium for cell culture and PBS is preferable. As the culture medium for cell culture, a known culture medium can be appropriately selected and used or after being appropriately modified as necessary, in consideration of the kind of the cells to be suspended. Specifically, the culture medium for cell culture may be, for example, MEM, α-MEM, D'MEM (Dulbecco's modified Eagle's medium), IMDM, HamF10, HamF12, Medium 199, RPMI 1640, RITC80-7, MCDB 104, MCDB 105, MCDB 153, MCDB 201, MCDB 202, Fisher's medium, or a mixed culture medium thereof. D'MEM is more preferable. In addition, in a case where the DMSO-containing cell composition is used as an effective component of a pharmaceutical composition, in order to suppress a rejection reaction in an animal to which a pharmaceutical composition containing the DMSO-containing cell composition is administered, the culture medium for cell culture is preferably a culture medium that does not contain animal proteins such as a serum component.

The number of cells contained in the DMSO-containing cell composition is not particularly limited, but is preferably 5×10⁶ cells/mL or more, more preferably 1×10⁷ cells/mL or more, still more preferably 2.5×10⁷ cells/mL or more, and even still more preferably 1×10⁸ cells/mL or more. In a case where the cell density in the composition is in the above-described ranges, the survival rate of cells and the tissue adhesion rate can be further improved. The upper limit value of the number of cells to be contained in the DMSO-containing cell composition is not particularly limited, but the number can be set to, for example, 1×10⁹ cells/mL or less, preferably 7×10⁸ cells/mL or less, and more preferably 4×10⁸ cells/mL or less.

In a case of preparing a pharmaceutical composition in which the DMSO-containing cell composition is encapsulated in a fibrin gel, it is preferable that the DMSO-containing cell composition does not contain dextran and hydroxyethyl starch. Dextran and hydroxyethyl starch are known as effective components of a cryopreservation agent used in combination with DMSO, but they also have an inhibitory effect on the formation of a fibrin gel. Therefore, it is preferable that the DMSO-containing cell composition to be mixed with the pre-gelation solution in the method for producing a hydrogel cell composition according to the present invention does not contain both dextran and hydroxyethyl starch.

As the DMSO-containing cell composition which is the cell composition according to the present invention, from the viewpoint of sufficiently enhancing the survival rate of cells and the tissue adhesion rate, a composition obtained by diluting cells with a solution in which only DMSO and albumin are mixed with each other at predetermined concentrations in a solution in which cells can survive is preferable, a composition obtained by diluting cells with a solution in which only DMSO and albumin are mixed with each other at predetermined concentrations in a culture medium for cell culture, PBS, or a mixed solution of a culture medium for cell culture and PBS is more preferable, a composition obtained by diluting cells with a solution in which only DMSO and albumin are mixed with each other at predetermined concentrations in D'MEM, PBS, or a mixed solution of D'MEM and PBS is still more preferable, and a composition obtained by diluting cells with a solution in which only DMSO and albumin are mixed with each other at predetermined concentrations in D'MEM is particularly preferable. The DMSO-containing cell composition is more preferably a cell composition in which cells are suspended in D'MEM containing 5.0 v/v % or greater and 10.0 v/v % or less of DMSO and 2.0 w/v % or greater and 5.0 w/v % or less of albumin.

Since the damage due to the freezing and the thawing treatment is sufficiently suppressed, the DMSO-containing cell composition can be frozen and stored for a long period of time. The freezing of the DMSO-containing cell composition may be program freezing in which the cooling rate is set to be constant or may be performed by a rapid cooling method using a deep freezer, dry ice, or liquid nitrogen, and the freezing method can be appropriately selected and used from the freezing methods of typical cell suspensions. The DMSO-containing cell composition according to the present invention has characteristics of less damage to cells even in a case where a rapid cooling method using liquid nitrogen is used. The thawing of a frozen substance of the DMSO-containing cell composition can be performed by appropriately selecting and using a typical method of thawing a cell frozen substance such as thawing using a constant-temperature tank at room temperature or higher and 37° C. or lower.

The DMSO-containing cell composition may be administered to an animal as it is as a pharmaceutical composition, but it is preferable that a composition obtained by being diluted with a solution in which cells can survive is used as various pharmaceutical compositions including a cell composition for transplantation from the viewpoint that the DMSO concentration can be further sufficiently decreased. As the solution in which cells can survive, any of the solutions described above can be used. The DMSO-containing cell composition can be used for producing a pharmaceutical composition having both a satisfactory survival rate and a satisfactory tissue adhesion rate in a case of being used for transplantation, only by being appropriately diluted with a solution in which cells can survive or by being used as it is without performing a treatment of removing DMSO. In the pharmaceutical composition containing the DMSO-containing cell composition, the DMSO concentration is preferably in a range of 2.5 v/v % or greater and 5.0 v/v % or less from the viewpoint that further improvement of the survival rate of cells and the tissue adhesion rate can be expected.

### <Hydrogel Cell Composition and Production Method Thereof>

In a case where the pharmaceutical composition containing a DMSO-containing cell composition as an effective component is a cell composition to be used for transplantation, it is preferable that the cells in the DMSO-containing cell composition are encapsulated in the scaffold material. Since the scaffold material is transplanted together with cells, a hydrogel which is a gel agent encapsulating water is preferable, and a biodegradable hydrogel is more preferable. Examples of such a hydrogel include a crosslinked polymer of polyvinyl alcohol to which cellulose, chitin, chitosan, collagen, fibrin, fibronectin, alginic acid, an alginate, carboxymethyl cellulose, a carboxymethyl cellulose salt, starch, gelatin, or polyethylene glycol is bonded.

In a case where a hydrogel cell composition in which cells in a DMSO-containing cell composition are encapsulated in a hydrogel is transplanted into an animal, the moisture in the hydrogel cell composition is rapidly replaced with a body fluid such as a synovial fluid after the transplantation, for example, in about 10 minutes or longer and 1 hour or shorter, the DMSO concentration is sufficiently decreased, and the cytotoxicity is reduced. That is, in a case where the hydrogel cell composition in which the DMSO-containing cell composition is encapsulated in a hydrogel is used as a pharmaceutical composition for transplantation, a pharmaceutical composition suitable for transplantation can be prepared without performing a treatment of removing DMSO from the DMSO-containing cell composition.

The pharmaceutical composition in which the cells in the DMSO-containing cell composition are encapsulated in a hydrogel can be produced by mixing the DMSO-containing cell composition with the hydrogel, but in order to encapsulate the cells more inside the hydrogel, a hydrogel to be gelated by mixing two or more kinds of pre-gelation solutions is preferable. Examples of such pre-gelation solutions include a combination of fibrinogen and thrombin, a combination of an alginate and a polyvalent metal ion solution, a combination of carboxymethyl cellulose and a crosslinking agent, a combination of chitosan and a pH adjusting solution, a combination of collagen and a pH adjusting solution, and a combination of polyethylene glycol and polyvinyl alcohol.

In the present invention, as the hydrogel encapsulating cells in the DMSO-containing cell composition, a hydrogel used as an adhesive for a biological tissue can be used. Specific examples thereof include fibrin glue (fibrin gel), a gelatin-resorcinol-polyfunctional aldehyde-based hydrogel, a cyanoacrylate-based hydrogel, a collagen gel, and a polyethylene glycol crosslinked polyvinyl alcohol gel. In the present invention, from the viewpoints of high safety, wide use, and satisfactory adhesiveness to cartilage tissues, it is preferable to use a fibrin gel, a collagen gel, or a polyethylene glycol crosslinked polyvinyl alcohol gel and more preferable to use a fibrin gel. The fibrin gel is a hydrogel in which fibrin synthesized by the decomposition of fibrinogen by thrombin is formed in a mesh shape.

The method for producing a hydrogel cell composition according to the present invention is a method for producing a hydrogel cell composition in which a DMSO-containing cell composition is encapsulated in a fibrin gel, and the method includes a step of mixing the DMSO-containing cell composition, fibrinogen, and thrombin to prepare a hydrogel cell composition. The DMSO-containing cell composition, fibrinogen, and thrombin are mixed with each other to form a fibrin gel to encapsulate the DMSO-containing cell composition. Since the formation of the fibrin gel is initiated by mixing the fibrinogen and the thrombin, in the present invention, it is preferable that the DMSO-containing cell composition and the thrombin are first mixed, and the obtained mixture is mixed with the fibrinogen. The DMSO-containing cell composition may be mixed with fibrinogen, and the obtained mixture may be mixed with thrombin.

The fibrinogen and the thrombin mixed with the DMSO-containing cell composition may be in the form of a dry powder, but it is preferable that the fibrinogen and thrombin are mixed with the DMSO-containing cell composition as a solution obtained by dissolving each of the fibrinogen and thrombin in an appropriate solvent. In a case where the fibrinogen and thrombin are mixed with each other in a state of a solution, the mixture is likely to be uniformly mixed with the DMSO-containing cell composition, and the DMSO concentration of the hydrogel cell composition to be finally produced can be further decreased. For example, in a case where Y mL of a thrombin solution is mixed with X mL of the DMSO-containing cell composition and Z mL of a fibrinogen solution is mixed with the obtained mixture (X, Y, and Z represent a positive number and preferably a natural number), the DMSO concentration of the produced hydrogel cell composition ([X+Y+Z] mL) is diluted to X/(X+Y+Z) times of the DMSO-containing cell composition.

It is preferable that the DMSO concentration of the hydrogel cell composition decreases from the viewpoint of suppressing cytotoxicity derived from DMSO. Meanwhile, in a case where the cell density of the hydrogel cell composition is somewhat high, the cell proliferation properties and tissue adhesiveness after transplantation tend to be enhanced. In the present invention, in a case where Y mL of a thrombin solution is mixed with X mL of the DMSO-containing cell composition, and Z mL of a fibrinogen solution is mixed with the obtained mixture (X represents a positive number, and Y and Z independently represent a number of 0 or greater), the amount of the DMSO-containing cell composition is preferably 30 v/v % or greater and 100 v/v % or less with respect to the total amount ([X+Y+Z] mL) of the produced hydrogel cell composition. In a case where fibrinogen and thrombin are mixed with the DMSO-containing cell composition as a dry powder, Y=Z=0 is satisfied, and the amount ratio of the DMSO-containing cell composition to the hydrogel cell composition is 100 v/v %. Here, it is preferable that the DMSO-containing cell composition, the thrombin solution, and the fibrinogen solution are prepared and mixed so that the amount ratio of the DMSO-containing cell composition to the hydrogel cell composition is 40 v/v % or greater and 60 v/v % or less and preferably 45 v/v % or greater and 55 v/v % or less. Since the DMSO-containing cell composition is diluted with a pre-gelation solution (the thrombin solution and the fibrinogen solution), the DMSO concentration of the obtained hydrogel cell composition is less than the DMSO concentration of the original DMSO-containing cell composition. A hydrogel cell composition in which the DMSO concentration with respect to the total amount of the composition is 2.0 v/v % or greater and 6.0 v/v % or less can be produced by mixing the mixture in the above-described manner.

As the thrombin solution, for example, a solution obtained by dissolving 100 units/mL or greater and 500 units/mL or less of thrombin in purified water can be used. As the fibrinogen solution, for example, a solution obtained by dissolving 10 mg/mL or greater and 200 mg/mL or less of fibrinogen in purified water can be used. As both the thrombin and the fibrinogen, a purified product purified from animal blood or a synthetic product artificially synthesized by using a microbial expression system or the like may be used.

The factor XIII and calcium ions are coagulation factors that strengthen the fibrin mesh structure. In addition, the fibrin gel in the cell composition for transplantation is decomposed by plasmin in the tissues of the transplantation destination, but aprotinin has an action of inhibiting plasmin. Therefore, each one of these may be dissolved in a thrombin solution or a fibrinogen solution. For example, the thrombin solution can contain calcium chloride, and the fibrinogen solution can contain the factor XIII and aprotinin. In addition, as the thrombin solution and the fibrinogen solution to be mixed with the DMSO-containing cell composition, a tissue adhesive used in clinical practice can also be used.

In the production of the hydrogel cell composition, the DMSO-containing cell composition, the thrombin solution, and the fibrinogen solution can be mixed by using, for example, a member for mixing two liquids, which includes a first injection port, a second injection port, a mixing portion that mixes a solution injected from the first injection port and a solution injected from the second injection port, and a discharge port that discharges the contents of the mixing portion. In a case where the member for mixing two liquids is used, for example, a mixed solution of the DMSO-containing cell composition and the thrombin solution is injected from the first injection port, and the fibrinogen solution is injected from the second injection port. The mixed solution of the DMSO-containing cell composition and the thrombin solution and the fibrinogen solution are mixed in the mixing portion to prepare a cell composition for transplantation. The cell composition for transplantation can be discharged from the discharge port and transplanted into target tissues.

A hydrogel cell composition in which the DMSO-containing cell composition is encapsulated in a fibrin gel can be prepared simply by mixing the DMSO-containing cell composition, the thrombin solution, and the fibrinogen solution. As described above, since the DMSO-containing cell composition according to the present invention causes less damage to cells even after thawing, the hydrogel cell composition prepared from the DMSO-containing cell composition can be transplanted into an animal even after the frozen substance of the DMSO-containing cell composition is thawed and allowed to stand at room temperature for 20 minutes or longer, preferably 20 minutes to 2 hours, more preferably 20 minutes to 1 hour, and still more preferably 20 minutes. For example, other operations and the like can also be performed after the DMSO-containing cell composition is subjected to the thawing treatment in an operating room by using the method for producing a hydrogel cell composition according to the present invention. In addition, in the hydrogel cell composition encapsulated in the fibrin gel, the cells are stably maintained for a relatively long period of time. Therefore, for example, even in a case where the formed hydrogel cell composition is stored at room temperature for several hours from the production to the transplantation, the cell survival rate after the transplantation is not significantly affected.

In a case where the DMSO-containing cell composition is cryopreserved, damage to cells can be suppressed by quickly carrying out the thawing. Therefore, it is preferable that the frozen DMSO-containing cell composition is thawed within 5 minutes.

In a case where the hydrogel cell composition produced by the method for producing a hydrogel cell composition according to the present invention is transplanted into an animal, both the survival rate of cells and the tissue adhesion rate are satisfactory. Therefore, the hydrogel cell composition is suitable as the pharmaceutical composition used for treatments of various diseases. For example, in a case where the hydrogel cell composition containing cartilage cells or mesenchymal stem cells, which is produced by the method for producing a hydrogel cell composition according to the present invention, is transplanted into a cartilage defect part, the cartilage of the defect part is regenerated, and a pathological condition caused by the deficiency can be improved.

### EXAMPLES

Hereinafter, the present invention will be described in more detail based on the examples, but the present invention is not limited to the following examples.

### <Cells>

Polydactyly-derived synovial cartilage cells (JCRB1693, obtained from JCRB Cell Bank, 8th subculture) were cultured in a D'MEM medium containing 10 v/v % of fetal bovine serum (FBS) using a flask. The cells that had been subcultured twice (the 10th subculture) were detached with trypsin, and 9.8×10⁷ cells were obtained as the number of live cells, which were referred to as “cultured cartilage cells”.

The cartilage tissues obtained from the patient with knee osteoarthritis were treated with collagenase to separate the cartilage cells. The recovered cartilage cells were cultured in a D'MEM medium containing 10 v/v % of FBS using a flask. The cultured cells were detached with trypsin, and 1.4×10⁸ cells were obtained as the number of live cells, which were referred to as “osteoarthritic patient-derived cartilage cells”.

Human adipose tissues collected from a Japanese person were treated with collagenase to separate human adipose-derived mesenchymal stem cells. The recovered human adipose-derived mesenchymal stem cells were cultured in an R:STEM medium (manufactured by ROHTO Pharmaceutical Co., Ltd.) using a flask (4th subculture). The cultured cells were detached with trypsin, and 1.4×10⁸ cells were obtained as the number of live cells, which were referred to as “adipose tissue-derived mesenchymal stem cells”.

### Example 1

The survival rate after thawing was examined for a frozen substance of a cell composition prepared with a cryopreservation agent having a different DMSO concentration and a different albumin concentration.

### (1) Preparation of Cryopreservation Liquid

Albumin (product number: 014-21543, manufactured by FUJIFILM Wako Pure Chemical Corporation) was added to a solution in which DMSO (product number: Cryoserv NDM-50, manufactured by Nipro Corporation) and a D'MEM medium (product number: Gibco 11971025, manufactured by Thermo Fisher Scientific, Inc.) were mixed in the blending amounts listed in the columns of “preparation method” in Table 1, thereby preparing a cryopreservation liquid having a composition listed in the columns of “composition” in Table 1.

TABLE 1

| | Composition | | | | |
|---|---|---|---|---|---|
| | Albumin | DMSO | Preparation method | | |
| | concentration | concentration | Albumin | DMSO | D'MEM |
| | (w/v %) | (v/v %) | (mg) | (μL) | (μL) |
| Cryopreservation | 2.5 | 2.5 | 50 | 50 | 1950 |
| liquid 1 | | | | | |
| Cryopreservation | 2.5 | 5.0 | 50 | 100 | 1900 |
| liquid 2 | | | | | |
| Cryopreservation | 5.0 | 5.0 | 100 | 100 | 1900 |
| liquid 3 | | | | | |
| Cryopreservation | 2.5 | 7.5 | 50 | 150 | 1850 |
| liquid 4 | | | | | |
| Cryopreservation | 5.0 | 7.5 | 100 | 150 | 1850 |
| liquid 5 | | | | | |
| Cryopreservation | 2.5 | 10.0 | 50 | 200 | 1800 |
| liquid 6 | | | | | |
| Cryopreservation | 5.0 | 10.0 | 100 | 200 | 1800 |
| liquid 7 | | | | | |

### (2) Freezing of Cells

1.3×10⁷ cells of cultured cartilage cells or osteoarthritic patient-derived cartilage cells were suspended in each of 500 μL of cryopreservation liquids 1 to 7, vials for cryopreservation (“NUNC CRYOTUBE 2 mL 368632”, manufactured by Thermo Fisher Scientific, Inc.) were filled with the total amount of each of the obtained suspensions, and the vials were stored at −80° C. for one day. Thereafter, the vials were moved to an environment in a liquid nitrogen gas phase and stored.

### (3) Evaluation of Survival Rate

The frozen cell suspension 1 day after the storage in the liquid nitrogen gas phase was maintained in a water bath at 37° C. for 1.5 minutes to be thawed. After the thawing, the suspension was maintained at room temperature for 20 minutes, 500 μL of D'MEM was added thereto, and the solution was maintained at 37° C. for 2 hours and 40 minutes. 50 μL of the cell suspension after the maintaining was collected each time, and the number of dead cells and the total number of cells were measured using a cell measuring device (“NucleoCounter NC-100”, manufactured by ChemoMetec A/S) (test sections 1 to 7). Based on the measurement results of the cell number, the survival rate (%) of each test section was calculated and evaluated using the following calculation formula. The results thereof are listed in Table 2.

$$[ Survival rate ( \% ) ] = ( [ total number of cells ] - [ number of dead cells ] ) / [ total number of cells ] \times 100 ( \% )$$

In addition, for comparison, a cell suspension in which 1.3×10⁷ cells of the cultured cartilage cells or the osteoarthritic patient-derived cartilage cells were suspended in 500 μL of D'MEM and stored at 4° C. for 2 days was used as a non-freezing group. Even for the non-freezing group, the survival rate was calculated by adding 500 μL of D'MEM after the suspension was maintained at room temperature for 20 minutes, maintaining the solution at 37° C. for 2 hours and 40 minutes, and counting the number of dead cells and the total number of cells (test section 8), in the same manner as the test group. The results thereof are listed in Table 2. In the table, “N. D.” denotes that no experiment had been carried out and thus there was no data.

TABLE 2

| | | Composition | | Survival rate (%) | |
|---|---|---|---|---|---|
| | | Albumin | DMSO | | Osteoarthritis |
| | Cryopreservation | concentration | concentration | Cultured | patient-derived |
| | liquid | (w/v %) | (v/v %) | cartilage cells | cartilage cells |
| Test section 1 | Cryopreservation | 2.5 | 2.5 | N.D. | 21.1 |
| | liquid 1 | | | | |
| Test section 2 | Cryopreservation | 2.5 | 5.0 | 81.1 | 74.9 |
| | liquid 2 | | | | |
| Test section 3 | Cryopreservation | 5.0 | 5.0 | 79.1 | 86.0 |
| | liquid 3 | | | | |
| Test section 4 | Cryopreservation | 2.5 | 7.5 | 82.8 | 84.4 |
| | liquid 4 | | | | |
| Test section 5 | Cryopreservation | 5.0 | 7.5 | 84.0 | 88.2 |
| | liquid 5 | | | | |
| Test section 6 | Cryopreservation | 2.5 | 10.0 | 78.5 | 87.6 |
| | liquid 6 | | | | |
| Test section 7 | Cryopreservation | 5.0 | 10.0 | 90.5 | 90.5 |
| | liquid 7 | | | | |
| Test section 8 | — (Non-freezing group) | — | — | 72.4 | 79.4 |

As listed in Table 2, the survival rates of the test groups (test sections 2 to 7) in which the cryopreservation liquid having a DMSO concentration of 5.0 v/v % or greater and 10.0 v/v % or less and an albumin concentration of 2.5 w/v % or greater and 5.0 w/v % or less was used for both the cultured cartilage cells and the osteoarthritis patient-derived cartilage cells were equal to or greater than that of the non-freezing group (test section 8), and was confirmed to be sufficiently high.

### (4) Evaluation of Cell Adhesion Rate

Each of the frozen cell suspensions (test sections 1 to 7) one day after the storage in the liquid nitrogen gas phase was maintained in a water bath at 37° C. for 1.5 minutes to be thawed. After the thawing, the suspension was maintained at room temperature for 20 minutes, 500 μL of D'MEM was added thereto, and the solution was maintained at 37° C. for 2 hours and 40 minutes. The cell suspension after the maintaining was seeded in 10% FBS-containing D'MEM to have 5.7×10⁵ cells as the number of live cells using a 6-well plate flask and cultured in an atmosphere of 37° C. and 8% CO₂. The cells were washed with an HBSS buffer to remove non-adhesive cells one day after the start of the culture, and the adhesive cells were detached with a cell treatment reagent (“reagent B for NucleoCounter NC-100”, manufactured by Chemometec A/S). The total number of cells was measured from the obtained cell suspension using a cell measuring device (“NucleoCounter NC-100”, manufactured by ChemoMetec A/S). Based on the measurement results of the cell number, the cell adhesion rate (%) of each test section was calculated and evaluated using the following calculation formula. The results thereof are listed in Table 3.

$$[ Cell adhesion rate ( \% ) ] = [ number of adhesive cells ] / [ number of seeded cells ] \times 100 ( \% )$$

In addition, for comparison, even for the non-freezing group (test section 8) prepared as described above, the cell adhesion rate was calculated by maintaining the cells at room temperature for 20 minutes, adding 500 μL of D'MEM thereto, maintaining the cells at 37° C. for 2 hours and 40 minutes, seeding and culturing the cells in 10% FBS-containing D'MEM such that the number of live cells was 5.7×10⁵ cells, and measuring the total number of adhesive cells 1 day after the start of the culture, in the same manner as the test group. The results thereof are listed in Table 3. In the table, “N. D.” denotes that no experiment had been carried out and thus there was no data.

TABLE 3

| | | Composition | | Cell adhesion rate (%) | |
|---|---|---|---|---|---|
| | | Albumin | DMSO | Cultured | Osteoarthritis |
| | Cryopreservation | concentration | concentration | cartilage | patient-derived |
| | liquid | (w/v %) | (v/v %) | cells | cartilage cells |
| Test section 1 | Cryopreservation | 2.5 | 2.5 | N.D. | N.D. |
| | liquid 1 | | | | |
| Test section 2 | Cryopreservation | 2.5 | 5.0 | 71.4 | 44.0 |
| | liquid 2 | | | | |
| Test section 3 | Cryopreservation | 5.0 | 5.0 | 60.7 | 40.7 |
| | liquid 3 | | | | |
| Test section 4 | Cryopreservation | 2.5 | 7.5 | 76.3 | 52.3 |
| | liquid 4 | | | | |
| Test section 5 | Cryopreservation | 5.0 | 7.5 | 55.8 | 42.3 |
| | liquid 5 | | | | |
| Test section 6 | Cryopreservation | 2.5 | 10.0 | 64.0 | 53.3 |
| | liquid 6 | | | | |
| Test section 7 | Cryopreservation | 5.0 | 10.0 | 64.9 | 34.9 |
| | liquid 7 | | | | |
| Test section 8 | — (Non-freezing group) | — | — | 84.2 | 74.4 |

As listed in Table 3, in the cell suspensions after freezing and thawing in the test sections 2 to 7 in which the survival rate was sufficiently high, the cell adhesion rate was 30% or greater in all the cartilage cells, although the cell adhesion rate was not as high as that in the cell suspension in the test section 8 which was the non-freezing group. In particular, in a case of the cultured cartilage cells, the cell adhesion rate was the highest in the test section 4 using the cryopreservation liquid having a DMSO concentration of 7.5 v/v % and an albumin concentration of 2.5 w/v % and was the highest in the test section 2 using the cryopreservation liquid having a DMSO concentration of 5.0 v/v % and an albumin concentration of 2.5 w/v %. The cell adhesion rate of the osteoarthritic patient-derived cartilage cells was the highest in the test section 6 using a cryopreservation liquid having a DMSO concentration of 10.0 v/v % and an albumin concentration of 2.5 w/v %, and was the second highest in the test section 4 using a cryopreservation liquid having a DMSO concentration of 7.5 v/v % and an albumin concentration of 2.5 w/v %. As shown in the results, it was confirmed that the cell adhesion rate after the cryopreservation of the cells was higher in a case where the albumin concentration of the cryopreservation liquid was 2.5 w/v % than in a case where the albumin concentration of the cryopreservation liquid was 5.0 w/v %.

### Example 2

The survival rates of the cells immediately after thawing after the cryopreservation of the cells and the survival rates of the cells after storage at 37° C. after thawing were compared with each other for the cryopreservation liquid prepared using D'MEM, PBS, or a mixed solution of D'MEM and PBS as a solvent. The osteoarthritic patient-derived cartilage cells were used as the cells. The albumin concentration and the DMSO concentration of the cryopreservation liquid were set to be the same as those of the cryopreservation liquid 4 in which both the survival rate and the cell adhesion rate were satisfactory in Example 1.

### (1) Preparation of Cryopreservation Liquid

A cryopreservation liquid having the composition listed in the columns of “composition” in Table 4 was prepared by adding a 20 w/v % albumin solution (“Blood Donation Albumin”, manufactured by KM Biologics Co., Ltd.) to a solution obtained by mixing DMSO (product number: Cryoserv NDM-50, manufactured by Nipro Corporation), a D'MEM medium (product number: Gibco 11971025, manufactured by Thermo Fisher Scientific, Inc.), and PBS (product number: Gibco 14190144, manufactured by Thermo Fisher Scientific, Inc.) in the blending amounts listed in the columns of “preparation method” in Table 4.

TABLE 4

| | Composition | | | | Preparation method | | | |
|---|---|---|---|---|---|---|---|---|
| | Albumin | DMSO | D'MEM | PBS | 20 w/v % | | | |
| | concentration | concentration | concentration | concentration | albumin | DMSO | D'MEM | PBS |
| | (w/v %) | (v/v %) | (v/v %) | (v/v %) | (mL) | (mL) | (mL) | (mL) |
| Cryopreservation | 2.5 | 7.5 | 80 | 0 | 0.625 | 0.375 | 4.0 | 0 |
| liquid 1 | | | | | | | | |
| Cryopreservation | 2.5 | 7.5 | 50 | 30 | 0.625 | 0.375 | 2.5 | 2.5 |
| liquid 2 | | | | | | | | |
| Cryopreservation | 2.5 | 7.5 | 0 | 80 | 0.625 | 0.375 | 0 | 4.0 |
| liquid 3 | | | | | | | | |

### (2) Freezing of Cells

2.9×10⁷ cells of the osteoarthritic patient-derived cartilage cells were suspended in 1 mL of a cryopreservation liquid, a vial for cryopreservation (“NUNC CRYOTUBE 2 mL 368632”, manufactured by Thermo Fisher Scientific, Inc.) was filled with the total amount of the suspension, and the vial was stored at −80° C. for one day. Thereafter, the vials were moved to an environment in a liquid nitrogen gas phase and stored.

### (3) Evaluation of Survival Rate

The frozen cell suspension stored at −150° C. or lower for 2 days was maintained in a water bath at 37° C. for 1.5 minutes to be thawed. After the thawing, the suspension was diluted to twice by adding the same amount of D'MEM. A part of the diluted cell suspension was fractionated to be used for measuring the survival rate immediately after the thawing, and the remaining part was maintained at 37° C. for 1 hour and used for measuring the survival rate after the maintaining. 40 μL of the cell suspension after the maintaining was collected each time, and the number of dead cells and the total number of cells were measured using a cell measuring device (“NucleoCounter NC-202”, manufactured by ChemoMetec A/S) (test sections 1 to 3). Based on the measurement results of the number of cells, the survival rate (%) of each test section was calculated and evaluated in the same manner as in Example 1. The results thereof are listed in Table 5.

TABLE 5

| | | Composition of | | | |
|---|---|---|---|---|---|
| | | cryopreservation liquid | | Survival rate (%) | |
| | | D'MEM | PBS | | 1 hour after |
| | Cryopreservation | concentration | concentration | Immediately | maintaining at |
| | liquid | (v/v %) | (v/v %) | after thawing | 37° C. |
| Test | Cryopreservation | 80 | 0 | 85 | 83 |
| section 1 | liquid 1 | | | | |
| Test | Cryopreservation | 50 | 30 | 83 | 82 |
| section 2 | liquid 2 | | | | |
| Test | Cryopreservation | 0 | 80 | 81 | 80 |
| section 3 | liquid 3 | | | | |

As listed in Table 5, it was confirmed that the survival rate after cryopreservation was not affected even in a case where the base material of the cryopreservation liquid was replaced with PBS instead of the culture medium for cell culture (for example, D'MEM). Specifically, even in a case where the base material of the cryopreservation liquid was replaced with PBS or a mixed solution of D'MEM and PBS from D'MEM, the cell survival rate of the cryopreserved cells stored at 37° C. for 1 hour after thawing was almost the same as the cell survival rate immediately after thawing.

### Example 3

The survival rate and the adhesion rate of the cells in a case where the cells after freezing and thawing are encapsulated in a fibrin gel were examined, and the influence of the composition of the cryopreservation liquid was examined. The osteoarthritic patient-derived cartilage cells were used as the cells.

### (1) Preparation of Cryopreservation Liquid

A cryopreservation liquid having the composition listed in the columns of “composition” in Table 6 was prepared by adding a 25 w/v % albumin solution (“Blood Donation Albumin”, manufactured by KM Biologics Co., Ltd.) to a solution obtained by mixing DMSO (product number: Cryoserv NDM-50, manufactured by Nipro Corporation) and a D'MEM medium (product number: Gibco 11971025, manufactured by Thermo Fisher Scientific, Inc.) in the blending amounts listed in the columns of “preparation method” in Table 6.

TABLE 6

| | Composition | | Preparation method | | |
|---|---|---|---|---|---|
| | Albumin | DMSO | 25 w/v % | | |
| | concentration | concentration | albumin | DMSO | D'MEM |
| | (w/v %) | (v/v %) | (mL) | (mL) | (mL) |
| Cryopreservation | 5.0 | 5.0 | 10.0 | 2.5 | 37.5 |
| liquid 1 | | | | | |
| Cryopreservation | 5.0 | 7.5 | 10.0 | 3.75 | 36.25 |
| liquid 2 | | | | | |
| Cryopreservation | 5.0 | 10.0 | 10.0 | 5.0 | 35.0 |
| liquid 3 | | | | | |

### (2) Freezing of Cells

6.0×10⁶ cells of the osteoarthritic patient-derived cartilage cells were suspended in 1.2 mL of a cryopreservation liquid, a vial for cryopreservation (“NUNC CRYOTUBE 2 mL 368632”, manufactured by Thermo Fisher Scientific, Inc.) was filled with the total amount of the suspension, and the vial was stored at −80° C. for one day.

### (3) Formation of Cell-Containing Fibrin Gel

A fibrinogen solution and a thrombin solution of a biological tissue adhesive (“BOLHEAL (registered trademark) for tissue adhesion”, manufactured by KM Biologics Co., Ltd.) were used for forming a fibrin gel.

A solution was prepared by adding 1 mL of a D'MEM medium to a fibrinogen powder (80 mg) of a 1 mL BOLHEAL preparation and mixing the solution, and this solution was used as a fibrinogen solution. Meanwhile, a solution was prepared by adding 0.75 mL of a D'MEM medium to a thrombin powder (250 units) of a 1 mL BOLHEAL preparation and mixing the solution, and this solution was used as a thrombin solution.

The frozen cell suspension stored at −80° C. for one day was maintained in a water bath at 37° C. for 1.5 minutes to be thawed. 50 μL of a thrombin solution was mixed with 450 μL of the cell suspension after thawing to prepare a thrombin-containing cell suspension. 100 μL of a fibrinogen solution was mixed with 100 μL of the thrombin-containing cell suspension, and 200 μL of a cell-containing fibrin gel was formed on a 48-well plate (manufactured by Corning Incorporated). 1.0 mL of a 10% FBS-containing D'MEM medium was added onto the cell-containing fibrin gel, and the cells were incubated in an atmosphere of 37° C. and 8% CO₂ for 24 hours.

### (4) Collection of Cells Carrying Fibrin

The gel mass in the well was recovered from the 48-well plate after incubation into a 50 mL centrifuge tube (product number: 352070, manufactured by Corning Incorporated), 1 mL of 0.05% collagenase (“Liberase MNP-S”, manufactured by F. Hoffmann-La Roche Ltd.) was added thereto, and the gel was digested at 37° C. for 6 hours. 1.0 mL of a 10% FBS-containing D'MEM medium was added to the digestive solution, and the solution was subjected to a centrifugal treatment (350 G, 5 minutes), the supernatant was removed, and the solution was resuspended in 1.0 mL of a 10% FBS-containing D'MEM medium, thereby obtaining a cell suspension.

### (5) Evaluation of Survival Rate

250 μL of the obtained cell suspension was collected each time, and the number of dead cells and the total number of cells were measured using a cell measuring device (“NucleoCounter NC-100”, manufactured by ChemoMetec A/S) (test sections 1 to 3). Based on the obtained results, the survival rate (%) of each test section was calculated and evaluated in the same manner as in Example 1. In addition, the survival rate was also measured for the cell suspension immediately after the thawing in the same manner as described above. The results thereof are listed in Table 7.

TABLE 7

| | Composition of | | | | Cell adhesion rate |
|---|---|---|---|---|---|
| | cryopreservation liquid | | Survival rate (%) | | (%) |
| | Albumin | DMSO | Immediately | After 24 hours of | After 24 hours of |
| | concentration | concentration | after | gelation + 6 hours of | gelation + 6 hours of |
| | (w/v %) | (v/v %) | thawing | dissolving treatment | dissolving treatment |
| Test | 5.0 | 5.0 | 99 | 64 | 46 |
| section 1 | | | | | |
| Test | 5.0 | 7.5 | 99 | 74 | 67 |
| section 2 | | | | | |
| Test | 5.0 | 10.0 | 97 | 63 | 69 |
| section 3 | | | | | |

### (6) Measurement of Cell Adhesion Rate

1.8 mL of a 10% FBS-containing D'MEM medium was mixed with 0.2 mL of the cell suspension that was recovered from the fibrin gel and resuspended, and the cells were seeded in a 6-well plate flask and cultured in an atmosphere of 37° C. and 8% CO₂. The cells were washed with an HBSS buffer to remove non-adhesive cells one day after the start of the culture, and the total number of adhesive cells was measured and the cell adhesion rate (%) was calculated in the same manner as in Example 1. The results thereof are listed in Table 7.

Since the fibrin gel was formed in the test sections 1 to 3, it was confirmed that the fibrin gel could be prepared by mixing the thrombin solution and the fibrin solution in a state of containing the cryopreservation liquid. In addition, as listed in Table 7, even in a case where the fibrin gel was maintained at the same temperature (37° C.) as in the body for 24 hours, the survival rate of the cartilage cells in the gel was 60% or greater, and the cell adhesion rate was also 40% or greater. As shown in these results, it was confirmed that the cartilage cells cryopreserved in a cryopreservation liquid having a DMSO concentration of 5.0 v/v % or greater and 10.0 v/v % or less could survive without losing the adhesion ability even in a case where the cells were transplanted into the body in a state of being encapsulated in a fibrin gel without removing DMSO.

### Reference Example 1

As a method of maintaining the freezing protective ability while reducing the DMSO component of the cryopreservation agent, a method of adding hydroxyethyl starch (HES) and dextran is known (Patent Documents 2 and 3). Therefore, a cryopreservation liquid containing hydroxyethyl starch and dextran was prepared, and the influence on the formation of the fibrin gel was examined.

### (1) Preparation of Cryopreservation Liquid

A cryopreservation liquid 1 (albumin concentration: 5.0 w/v %, DMSO concentration: 7.5 v/v %) was prepared by mixing 10 mL of a 25 w/v % albumin solution, 3.75 mL of DMSO, and 36.25 mL of a D'MEM medium.

A cryopreservation liquid 2 (albumin concentration: 4.0 w/v %, DMSO concentration: 5.0 v/v %, hydroxyethyl starch concentration: 6.0 w/v %) was prepared by mixing 3.2 mL of a 25 w/v % albumin solution, 6.8 mL of a HES solution (solution prepared by dissolving 6 g of hydroxyethyl starch (“CP-1 (registered trademark) High Grade”) and 5 mL of DMSO in physiological saline to have a volume of 34 mL), and 10.0 mL of a D'MEM medium.

A cryopreservation liquid 3 (albumin concentration: 5.0 w/v %, DMSO concentration: 7.5 v/v %, dextrose concentration: 2.0 w/v %, dextran concentration: 1.0 w/v %) was prepared by dissolving 8.0 mL of a 25 w/v % albumin solution, 0.8 g of dextrose, and 0.4 g of dextran (dextran 40) in 29.0 mL of a D'MEM medium adding 3.0 mL of DMSO to the solution, and mixing the solution.

### (2) Formation of Fibrin Gel

A fibrinogen solution and a thrombin solution were prepared in the same manner as in Example 3.

50 μL of the thrombin solution was added to 450 μL of a cryopreservation liquid and mixed to prepare a thrombin preservation solution. 500 μL of the thrombin preservation solution and 500 μL of the fibrinogen solution were mixed to obtain a fibrin mixture. About 200 μL of the obtained fibrin mixture was added dropwise onto a Petri dish and visually observed.

As a result of visual observation, it was confirmed that a transparent fibrin gel was formed in the fibrin mixture prepared from the cryopreservation liquid 1. Meanwhile, the fibrin mixture prepared from the cryopreservation liquid 2 or the fibrin mixture prepared from the cryopreservation liquid 3 was cloudy, and the fibrin gel was not formed. As shown in these results, it was confirmed that in a case where a cryopreservation liquid containing hydroxyethyl starch and dextran was used, a fibrin gel could not be formed from the cryopreservation liquid.

### Example 4

The cartilage-forming ability of the cartilage cells encapsulated in the fibrin gel after freezing and thawing was examined. Cultured cartilage cells were used as the cells.

### (1) Preparation of Cryopreservation Liquid

The cryopreservation liquid 1 having the composition listed in the columns of “composition” in Table 8 was prepared by adding albumin (rice-derived human recombinant albumin (powder), product number: 014-21543, manufactured by FUJIFILM Wako Pure Chemical Corporation) to a solution obtained by mixing DMSO (product number: Cryoserv NDM-50, manufactured by Nipro Corporation) and a D'MEM medium (product number: Gibco 11971025, manufactured by Thermo Fisher Scientific, Inc.) in the blending amounts listed in the columns of “preparation method” in Table 8.

TABLE 8

| | Composition | | | | |
|---|---|---|---|---|---|
| | Albumin | DMSO | Preparation method | | |
| | concentration | concentration | Albumin | DMSO | D'MEM |
| | (w/v %) | (v/v %) | (mg) | (mL) | (mL) |
| Cryopreservation | 2.5 | 7.5 | 250 | 0.75 | 9.25 |
| liquid 1 | | | | | |

### (2) Freezing of Cells

The cultured cartilage cells were suspended in 500 μL of the cryopreservation liquid 1, 460 μL of 1.6×10⁷ cells were prepared, a vial for cryopreservation (“NUNC CRYOTUBE 2 mL 368632”, manufactured by Thermo Fisher Scientific, Inc.) was filled with the cells, and the vial was stored at −80° C. for one day. Thereafter, the vials were moved to an environment in a liquid nitrogen gas phase and stored.

### (3) Formation of Cell-Containing Fibrin Gel

A fibrinogen solution and a thrombin solution were prepared in the same manner as in Example 3.

The cell suspension stored in a liquid nitrogen gas phase for 1 day was maintained in a water bath at 37° C. for 1.5 minutes to be thawed. 50 μL of a thrombin solution was mixed with 450 μL of the cell suspension after thawing to prepare a thrombin-containing cell suspension. 10 μL of the fibrinogen solution was mixed with 10 μL of the thrombin-containing cell suspension, and 20 μL of a cell-containing fibrin gel was formed in a 12-well plate.

### (4) Cartilage Cell Differentiation Method

3 mL of a differentiation-inducing culture medium (90 v/v % D'MEM/F12 (product number: 11320-033, manufactured by Thermo Fisher Scientific, Inc.), 10 v/v % FBS, 200 ng/mL IGF-1 (“Somazon injection 10 mg”, manufactured by OCEAN PACIFIC CO., LTD.), 400 ng/mL BMP-7 (product number: 354-BP-010/CF, manufactured by R&D SYSTEMS, Inc.), 10 mg/mL insulin (product number: 099-06473, manufactured by FUJIFILM Wako Pure Chemical Corporation), 100 mg/L ascorbic acid (product number: Vitamin C injection “Fuso”, 100 mg, manufactured by Fuso Pharmaceutical Industries, Ltd.), 400 ng/mL BMP-7 (product number: 354-BP-010/CF, manufactured by R&D SYSTEMS, Inc.)) was added to the cell-containing fibrin gel to culture the cells at 37° C. and 8% CO₂. The culture medium was exchanged at a frequency of once every 2 or 3 days from the start of the culture.

### (5) Safranin O Staining

The cell-containing fibrin gel was recovered after being cultured for 4 weeks and fixed with a fixing liquid (0.4% cetylpyridinium chloride (product number: C0732-100G, manufactured by Sigma-Aldrich Co., LLC), 4% paraformaldehyde (product number: 163-20145, manufactured by FUJIFILM Wako Pure Chemical Corporation)), thereby preparing a pathological tissue specimen. The pathological tissue specimen was stained with Safranin O, which is cartilage matrix-specific staining, and the degree of differentiation into cartilage cells was evaluated by cartilage tissue formation.

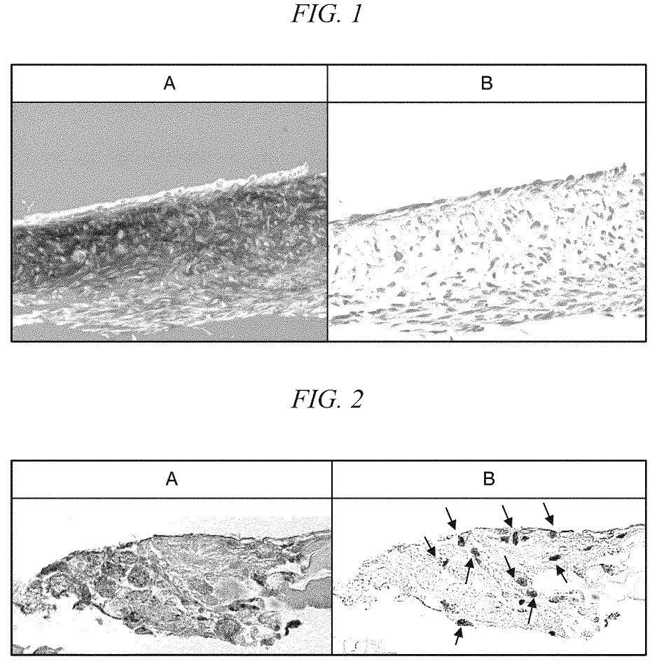

FIG. 1 shows Safranin O-stained images of the cell-containing fibrin gel 4 weeks after culture. FIG. 1(A) is a cartilage matrix-stained enhanced image, and FIG. 1(B) is a cell nucleus-enhanced image. The densely stained portion was shown in a region of the upper ⅔ or more of the tissue, and the formation of the cartilage tissue was confirmed. Since the formation of cartilage tissues was confirmed, it was speculated that the cartilage matrix was secreted from the fibrin gel-embedded cartilage cells, and it was confirmed that the function as the cartilage cells could be maintained even in a state where the cells were cryopreserved in a cryopreservation liquid having an albumin concentration of 2.5 w/v % and a DMSO concentration of 7.5 v/v %, thawed, and encapsulated in the fibrin gel.

### Example 5

The adipose tissue-derived mesenchymal stem cells were frozen and thawed to be encapsulated in a fibrin gel in the same manner as in Example 4, and the influence on the matrix secretion activity thereof was examined.

### (1) Preparation of Cryopreservation Liquid

The cryopreservation liquid 1 was prepared in the same manner as in Example 4.

### (2) Freezing of Cells

The adipose tissue-derived mesenchymal stem cells were suspended in 500 μL of the cryopreservation liquid 1, 460 L of 1.7×10⁷ cells were prepared, a vial for cryopreservation (“NUNC CRYOTUBE 2 mL 368632”, manufactured by Thermo Fisher Scientific, Inc.) was filled with the cells, and the vial was stored at −80° C. for one day in the same manner as in Example 4. Thereafter, the vials were moved to an environment in a liquid nitrogen gas phase and stored.

### (3) Formation of Cell-Containing Fibrin Gel

The cell suspension stored in the liquid nitrogen gas phase for 1 day was thawed and mixed with a thrombin solution and a fibrinogen solution in the same manner as in Example 4, and 20 μL of a cell-containing fibrin gel was formed in a 12-well plate.

### (4) Cartilage Cell Differentiation Method

3 mL of the differentiation-inducing culture medium used in Example 4 was added to the cell-containing fibrin gel, and the cells were cultured at 37° C. and 8% CO₂. The culture medium was exchanged at a frequency of once every 2 or 3 days from the start of the culture.

### (5) Evaluation of Matrix Secretion Activity

The cell-containing fibrin gel was recovered 4 weeks after culture, fixed with the fixing liquid used in Example 4, and embedded in paraffin, thereby preparing a paraffin tissue specimen. The paraffin tissue specimen was thinly sliced, and type I collagen immunostaining and nuclear staining were performed. The type I collagen immunostaining was performed using a rabbit anti-collagen I polyclonal antibody (1.0 mg/mL, manufactured by Abeam Limited), and the nuclear staining was performed using Mayer's hematoxylin.

FIG. 2 shows stained images of the type I collagen immunostaining and the nuclear staining of the cell-containing fibrin gel 4 weeks after culture. FIG. 2(A) shows a stained image with type I collagen, and FIG. 2(B) shows a stained image with hematoxylin staining in the same visual field. In FIG. 2(B), the arrows indicate cell nuclei. Tissues in which cell nuclei shown by the elliptical dense staining were present were stained with type I collagen. This type I collagen is considered to be a matrix secreted by the transplanted adipose tissue-derived mesenchymal stem cells, and it was confirmed that the function as the adipose tissue-derived mesenchymal stem cells can be maintained even in a state where the adipose tissue-derived mesenchymal stem cells are cryopreserved in a cryopreservation liquid having an albumin concentration of 2.5 w/v % and a DMSO concentration of 7.5 v/v %, thawed, and encapsulated in a fibrin gel.

### Example 6

The cartilage cell-containing fibrin gel produced in Example 4 was transplanted into a cartilage defect rabbit model, and the cartilage-forming ability was evaluated.

### (1) Preparation of Cryopreservation Liquid

The cryopreservation liquid 1 was prepared in the same manner as in Example 4.

### (2) Freezing of Cells

The cultured cartilage cells were suspended in the cryopreservation liquid 1, stored at −80° C. for 1 day, and moved to a liquid nitrogen gas phase for storage in the same manner as in Example 4.

### (3) Cartilage Defect Model Animal

An immunosuppressed state rabbit (immunosuppressed rabbit) was obtained by administering a dose of 2.4 mg of an immunosuppressant tacrolimus once a day from 2 days before transplantation to 7 days after transplantation and administering the same every other day after 7 days from the transplantation to a JW rabbit having a body weight of 3.0 kg. The transplantation was performed in a state where the immunosuppressed state was maintained.

Immediately before the transplantation, a cartilage defect having a diameter of 4 mm and a depth of 2 mm was provided in the knee cartilage of both legs of the immunosuppressed rabbit to prepare a cartilage defect model animal.

### (4) Formation and Transplantation of Cell-Containing Fibrin Gel

The cell suspension stored in a liquid nitrogen gas phase for 1 day was thawed in the same manner as in Example 4. 50 μL of a thrombin solution was mixed with 450 μL of the cell suspension after thawing to prepare a thrombin-containing cell suspension. 10 μL of a fibrinogen solution was added dropwise to the cartilage defect site of the cartilage defect model animal, 10 μL of a thrombin-containing cell suspension was added dropwise thereto, and both liquids were mixed quickly by pipetting at the defect site and allowed to stand for 5 minutes. After the formation of the gel after the standing was visually confirmed, the knee joint was sutured.

### (5) Safranin O Staining and HE Staining

A transplantation site where the cartilage cells were transplanted to the cartilage defect site was collected from the cartilage defect model animal four weeks after the transplantation to prepare a specimen. The specimen was fixed with the fixing liquid used in Example 4, decalcified in 10% EDTA·2Na (pH of 7.0 or greater and 7.4 or less) for about 4 weeks, and embedded in paraffin to prepare a paraffin tissue specimen. The paraffin tissue specimen was thinly sliced, stained with HE and Safranin O, and the degree of tissue repair by cartilage cell transplantation was evaluated by cartilage tissue evaluation.

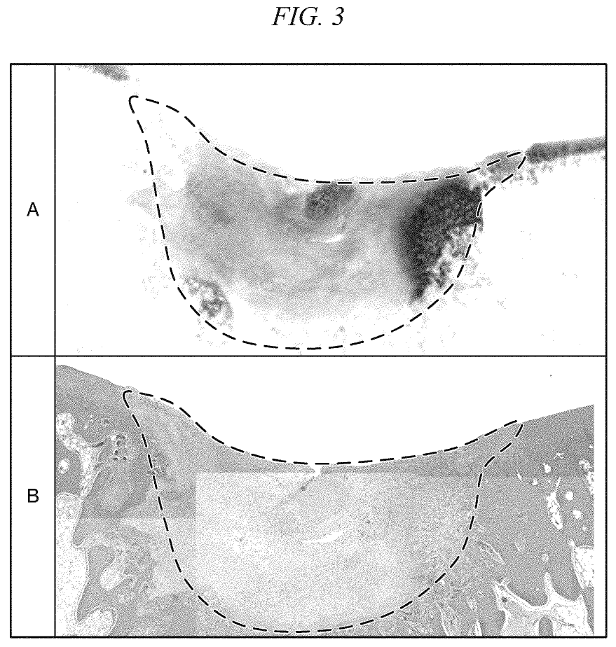

FIG. 3 shows a Safranin O-stained image and an HE-stained image of the transplantation site, in which the cells were collected from the cartilage defect model animal 4 weeks after the transplantation. FIG. 3(A) shows a Safranin O-stained image, and FIG. 3(B) shows an HE-stained image in the same visual field. In FIG. 3(B), a portion surrounded by the dotted line part indicates the cartilage defect part. The Safranin O-stained portion is shown corresponding to the regenerated part (dotted line part) of the cartilage defect, and it was confirmed that the transplantation site was filled with the cartilage matrix. As shown in the results, it was confirmed that, in a case where the cartilage cells cryopreserved in a cryopreservation liquid having an albumin concentration of 2.5 w/v % and a DMSO concentration of 7.5 v/v %, thawed, and transplanted in a state of being encapsulated in the fibrin gel was transplanted into the cartilage defect part, the transplanted cartilage cells adhered to the cartilage tissues to secrete a cartilage-specific matrix, and the defect was filled with the matrix.

### Example 7

The cryopreserved cell suspension was encapsulated in a fibrin gel, a polyvinyl alcohol (PVA)-polyethylene glycol (PEG) gel, or a collagen gel, and the cell survival rate of each dosage form was examined.

### (1) Cells

Polydactyly-derived synovial cartilage cells (JCRB1693, obtained from JCRB Cell Bank, 12th subculture) were cultured in a D'MEM medium containing 10 v/v % of fetal bovine serum (FBS) using a flask. The cultured cells (cells in the 13th subculture) were detached with trypsin, and 3.3×10⁸ cells were obtained as the number of live cells, which were referred to as “cultured cartilage cells”.

### (2) Preparation of Cryopreservation Liquid

A cryopreservation liquid having the composition listed in the columns of “composition” in Table 9 was prepared by adding a 20% albumin solution (rice-derived human recombinant albumin, product number: HYC002C02, manufactured by Wuhan Healthgen Biotechnology Corp.) to a solution obtained by mixing DMSO (product number: Cryoserv NDM-50, manufactured by Nipro Corporation) and a D'MEM medium (product number: Gibco 11971025, manufactured by Thermo Fisher Scientific, Inc.) in the blending amounts listed in the columns of “preparation method” in Table 9.

TABLE 9

| | Composition | | | | |
|---|---|---|---|---|---|
| | Albumin | DMSO | Preparation method | | |
| | concentration | concentration | 20% albumin solution | DMSO | D'MEM |
| | (w/v %) | (v/v %) | (μL) | (μL) | (mL) |
| Cryopreservation | 2.5 | 7.5 | 625 | 375 | 4.0 |
| liquid | | | | | |

### (3) Freezing of Cells

The cultured cartilage cells were suspended in a cryopreservation liquid, 1.2 mL of 3.4×10⁷ cells were prepared, and a vial for cryopreservation (frozen tube, manufactured by Nipro Corporation) was filled with the cells and stored at −80° C. for one day. Thereafter, the vials were moved to an environment in a liquid nitrogen gas phase and stored.

### (4) Formation of Cell-Containing Fibrin Gel

The cell suspension stored in a liquid nitrogen gas phase for one day or longer was maintained in a water bath at 37° C. for 1.5 minutes to be thawed. 180 μL of the suspension was collected from the thawed cell suspension and mixed with 20 μL of the thrombin solution prepared by the same method as in Example 3, thereby preparing a thrombin-containing cell suspension. 25 μL of the fibrinogen solution prepared by the same method as in Example 3 was mixed with 25 μL of the thrombin-containing cell suspension, and 50 μL of a cell-containing fibrin gel was formed on a glass bottom dish (diameter: 35 mm, glass surface diameter: 12 mm). 500 μL of a 10% FBS-containing D'MEM medium was added onto the cell-containing fibrin gel, and the cells were incubated in an atmosphere of 37° C. and 8% CO₂ for 1 day.

### (5) Formation of Cell-Containing PVA-PEG Gel

A reaction solution was prepared by mixing water, a 3D buffer of TrueGel3D Hydrogel Kits (TRUE5, manufactured by Sigma-Aldrich Co., LLC), and FAST-PVA in the blending amounts listed in the columns of “preparation method” in Table 10.

TABLE 10

| | Preparation method | | |
|---|---|---|---|
| | Water (μL) | 3D Buffer (μL) | FAST-PVA (μL) |
| Reaction solution | 13.6 | 2.4 | 2.0 |

9 μL of the cell suspension used in the item (4) was collected and mixed with the reaction solution. The reaction solution containing the cell suspension was mixed with PEG non cell-degradable crosslinker (TRUE5, manufactured by Sigma-Aldrich Co., LLC) which had been added dropwise (3 μL) onto a glass bottom dish (diameter: 35 mm, glass surface diameter: 12 mm), thereby forming a 30 μL cell-containing PVA-PEG gel. 500 μL of a 10% FBS-containing D'MEM medium was added onto the cell-containing PVA-PEG gel, and the cells were incubated in an atmosphere of 37° C. and 8% CO₂ for 1 day.

### (6) Formation of Cell-Containing Collagen Gel

A 30% FBS-containing D'MEM medium and bovine type I collagen (3 mg/mL, ASC-1-100-20, manufactured by Nippi Inc.) were mixed in the blending amounts listed in the columns of “production method” in Table 11 to prepare a collagen liquid.

TABLE 11

Preparation method

| | 30% FBS-containing | Bovine type |
|---|---|---|
| | D'MEM medium (μL) | I collagen (μL) |
| Collagen solution | 50 | 100 |

150 μL of the cell suspension used in the above (4) was collected, mixed with a collagen liquid (150 μL), and added dropwise to a glass bottom dish (diameter: 35 mm, glass surface diameter: 12 mm), and the cells were incubated in an atmosphere of 37° C. and 8% CO₂ for 20 minutes, thereby forming a 50 μL cell-containing collagen gel. 500 μL of a 10% FBS-containing D'MEM medium was added onto the cell-containing collagen gel, and the cells were incubated in an atmosphere of 37° C. and 8% CO₂ for 1 day.

### (7) Staining of Live Cells/Dead Cells and Fluorescence Observation

A Cellstain double staining kit (Dojindo Laboratories) was thawed. 20 μL of Calcein-AM (1 mmol/L) or 30 μL of PI (1.5 mmol/L) was added to 5 mL of DPBS, mixed, and a staining liquid (Calcein-AM (4 μmol/L) and PI (9 mol/L)) was prepared.

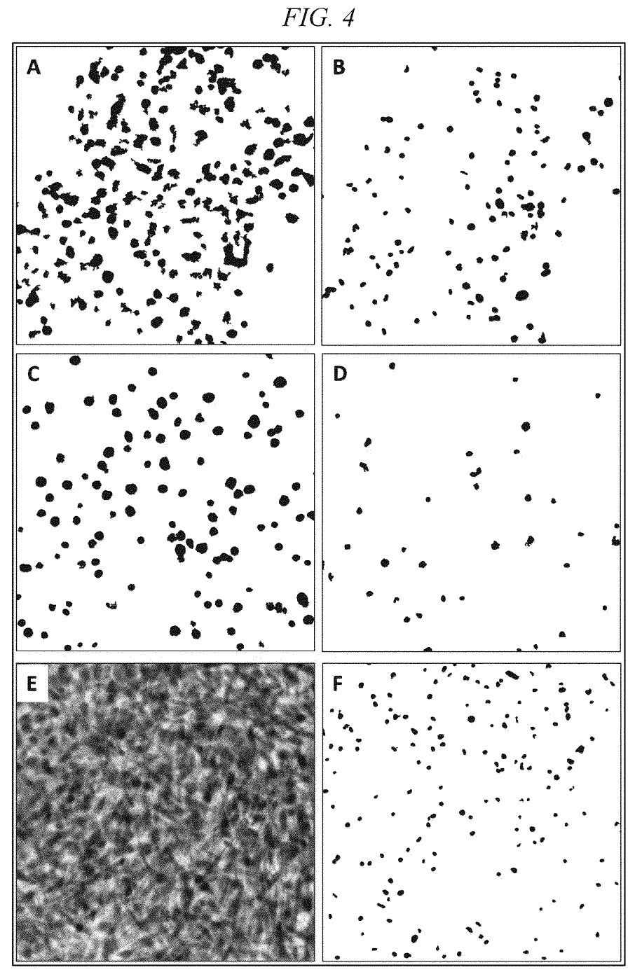

The culture solution was removed from the cell-containing fibrin gel, the cell-containing PVA-PEG gel, and the cell-containing collagen gel formed on the glass bottom dish, and the cells were washed by being allowed to stand in 500 μL of DPBS at room temperature for 20 minutes. The cells were washed twice. Thereafter, the cells were subjected to double staining with Calcein-AM and PI using a Cellstain double staining kit (manufactured by Dojindo Laboratories) and imaged with a fluorescence microscope (BX50, manufactured by OLYMPUS Corporation). From the obtained images, it was confirmed that the number of live cells was greater than or equal to the number of dead cells in all the gels. Therefore, it was confirmed that the fibrin gel, the PVA-PEG gel, and the collagen gel can be used for encapsulating the cartilage cells. (FIG. 4).

### Example 8

The drug efficacy of the cartilage cells after the cryopreservation was evaluated by the redifferentiation ability of the alginate gel culture.

### (1) Cells

The cartilage tissues obtained from a patient with polydactyly were treated with collagenase, and the cartilage cells were separated and cultured in a D'MEM medium containing 10 v/v % of FBS using a flask. The cells that had been subcultured three times (the cells in the fourth subculture) were detached with trypsin to obtain cartilage cells (P4 cartilage cells). The cells were cultured in the same manner as described above to prepare cells in the 7th subculture (P7 cartilage cells), cells in the 10th subculture (P10 cartilage cells), and cells in the 11th subculture (P11 cartilage cells), and cryopreserved using the cryopreservation liquid prepared by the same method as in Example 7.

### (2) Culturing of Alginate Gel (Beads)

The P4 cartilage cells, the P7 cartilage cells, the P10 cartilage cells, and the P11 cartilage cells stored in a liquid nitrogen gas phase for 1 day or longer were maintained in a water bath at 37° C. for 1.5 minutes to be thawed. A sodium alginate solution (ABC-AL, PG Research, Inc.) was added so that the live cell concentration was 2×10⁶ cells/mL, the solution was stirred with a pipette, a calcium chloride solution (ABC-CA, manufactured by PG Research, Inc.) was added thereto, and granular alginate beads were prepared by dropwise addition.

The alginate beads containing cells were transferred to a 6-well plate with a target density of 100 beads/well or less. 5 mL of the differentiation-inducing culture medium used in Example 4 was added thereto, and the cells were cultured at 37° C. and 8% CO₂. During the culture period, the culture medium was exchanged at a frequency of once every 2 or 3 days.

### (3) Recovery of Cells and Measurement of Gene Expression

On the 21st day after the preparation of the beads, about 50 beads were recovered from the 6-well plate, and 30 mL of a sodium citrate solution (ABC-CI, manufactured by PG Research, Inc.) was added thereto to completely dissolve the alginate beads. The dissolved solution was divided into two parts, and physiological saline was added to each part. After centrifugation at 350×g for 3 minutes, the supernatant was aspirated and discarded, an appropriate amount of PBS was further added thereto, and the supernatant was aspirated and discarded after centrifugation at 350×g for 3 minutes. 250 μL of a lysis buffer was added thereto, and the cells were dissolved by stirring the solution to prepare a dissolved solution.

mRNA was extracted from the dissolved solution using NucleoSpin RNA Plus (manufactured by Takara Bio Inc.), and cDNA was synthesized using Super Script VILO Master Mix (manufactured by Thermo Fisher Scientific, Inc.). The cDNA was mixed with ddPCR Supermix for Probes No dUTP (manufactured by Bio-Rad Laboratories, Inc.) and a primer (Thermo Fisher Scientific, Inc.) and droplet generator oil (manufactured by Bio-Rad Laboratories, Inc.), and the mRNA expression level was quantified with QX200 (manufactured by Bio-Rad Laboratories, Inc.).

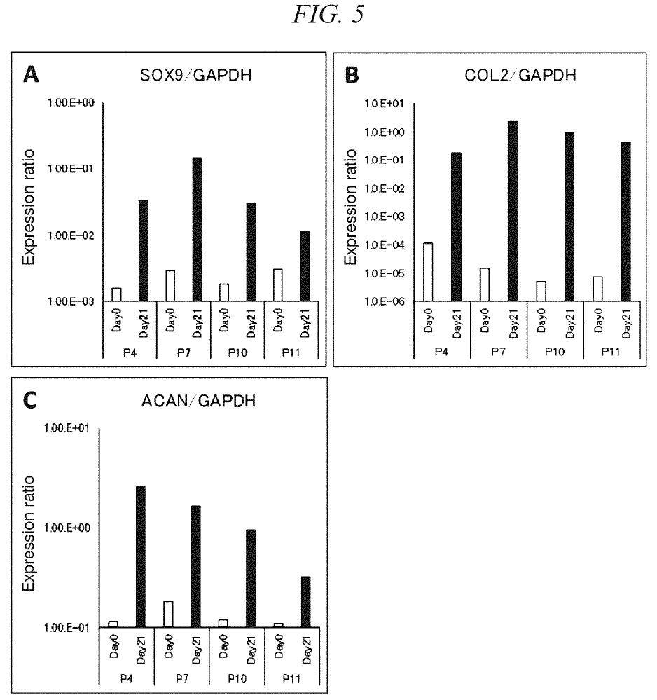

The expression ratio of each of SOX9, which is a cartilage transcription factor, and COL2 and ACAN, which are cartilage matrix (differentiation) markers, to GAPDH is shown in FIG. 5. As shown in FIG. 5, the mRNA expression ratio after 21 days from the differentiation initiation day was increased in all the cells of all the subcultures (subculture numbers 4, 7, 10, and 11). As shown in the results, it was confirmed that the cartilage cells frozen using the cryopreservation liquid according to the present invention maintained the differentiation ability. Therefore, it was suggested that the cartilage cells frozen using the cryopreservation liquid according to the present invention can effectively function as cartilage cells even after the transplantation.

1. A cell composition,
wherein a cell is suspended in a solution containing 5.0 v/v % or greater and 10.0 v/v % or less of dimethyl sulfoxide and 2.0 w/v % or greater and 5.0 w/v % or less of albumin.

2. The cell composition according to claim 1,
wherein a concentration of the dimethyl sulfoxide in the solution is 5.0 v/v % or greater and 7.5 v/v % or less.

3. The cell composition according to claim 1,
wherein a concentration of the dimethyl sulfoxide in the solution is 7.5 v/v %.

4. The cell composition according to claim 1,
wherein a concentration of the albumin in the solution is 2.5 w/v %.

5. The cell composition according to claim 1,
wherein the cell is frozen.

6. The cell composition according to claim 1,
wherein the cell is a cartilage cell or a mesenchymal stem cell.

7. The cell composition according to claim 1,
wherein the cell composition does not contain dextran.

8. The cell composition according to claim 1,
wherein the solution is a solution obtained by allowing a culture medium for cell culture, phosphate buffered saline, or a mixed solution thereof to contain dimethyl sulfoxide and albumin.

9. (canceled)

10. The cell composition according to claim 1,
wherein the cell is suspended in D'MEM containing 5.0 v/v % or greater and 10.0 v/v % or less of dimethyl sulfoxide and 2.0 w/v % or greater and 5.0 w/v % or less of albumin.

11. The cell composition according to claim 1,
wherein a concentration of the cell in the cell composition is 2.5×10⁷ cells/mL or greater.

12. A pharmaceutical composition comprising:
the cell composition according to claim 1.

13. (canceled)

14. The pharmaceutical composition according to claim 12,
wherein the cell composition is encapsulated in a hydrogel.

15. The pharmaceutical composition according to claim 14,
wherein the hydrogel is a fibrin gel.

16. The pharmaceutical composition according to claim 12,
wherein the pharmaceutical composition is a composition for transplanting into a cartilage tissue.

17. (canceled)

18. (canceled)

19. A method for producing a hydrogel cell composition, comprising:
a step of mixing a cell composition containing dimethyl sulfoxide, albumin, and a cell, fibrinogen, and thrombin to prepare a hydrogel cell composition,
wherein the cell composition contains 5.0 v/v % or greater and 10.0 v/v % or less of dimethyl sulfoxide, 2.0 w/v % or greater and 5.0 w/v % or less of albumin, and one or more cells selected from the group consisting of a cartilage cell and an adipose-derived mesenchymal stem cell.

20. (canceled)

21. The method for producing a hydrogel cell composition according to claim 19,
wherein the cell composition is formed such that the cell is suspended in a solution obtained by allowing a culture medium for cell culture, phosphate buffered saline, or a mixed solution thereof to contain dimethyl sulfoxide and albumin.

22. The method for producing a hydrogel cell composition according to claim 21,
wherein the culture medium for cell culture is D'MEM.

23. (canceled)

24. The method for producing a hydrogel cell composition according to claim 19,
wherein a member which mixes two liquids, which includes a first injection port, a second injection port, a mixing portion that mixes a solution injected from the first injection port and a solution injected from the second injection port, and a discharge port that discharges contents of the mixing portion, is used to
inject a mixture of the cell composition and the thrombin from the first injection port and inject the fibrinogen from the second injection port, and
in the mixture, the mixture of the cell composition and the thrombin is mixed with the fibrinogen to prepare a hydrogel cell composition, and the hydrogel cell composition is discharged from the discharge port.

25. The method for producing a hydrogel cell composition according to claim 19,
wherein the cell composition is a frozen substance, and
the hydrogel cell composition is prepared by thawing the cell composition, mixing the cell composition with thrombin, and mixing the obtained mixture with fibrinogen.

26. The method for producing a hydrogel cell composition according to claim 19,
wherein the cell composition is thawed within 5 minutes, and
the hydrogel cell composition is transplanted into an animal after 20 minutes to 2 hours from the thawing of the cell composition.
